(19) United States
(12) Patent Application Publication
(10) Pub. No.: US 2019/0300484 A1
(43) Pub. Date: Oct. 3, 2019

(54) AN IMPROVED PROCESS FOR THE PREPARATION OF REGORAFENIB

(71) Applicant: NATCO PHARMA LTD, Hyderabad (IN)

(72) Inventors: Pulla Reddy MUDDASANI, Hyderabad (IN); Shankar Reddy BUDIDETI, Hyderabad (IN); Srinivasachary CHINTALAPATI, Hyderabad (IN); Tirupathi KOTTE, Hyderabad (IN); Sambasiva Rao TALASILA, Hyderabad (IN); Venkaiah Chowdary NANNAPANENI, Hyderabad (IN)

(21) Appl. No.: 16/070,759

(22) PCT Filed: Mar. 31, 2016
(86) PCT No.: PCT/IN2016/050099

§ 371 Date: Jul. 17, 2018

(30) Foreign Application Priority Data
Jan. 18, 2016 (IN) 201641001756

(51) Int. Cl. C07D 213/81

(52) U.S. Cl. CPC C07D 213/81

(57) ABSTRACT

The present invention relates to an improved process for the preparation of Regorafenib with genotoxic impurities at well below threshold limit and high yield. The present invention also relates to an improved process for the preparation of regorafenib form-I with high purity.

### FIELD OF THE INVENTION

The present invention relates to a commercially cost effective process for the preparation of Regorafenib with high purity and high yield. The present invention also relates to an improved process for the preparation of regorafenib form-I with high purity.

### BACKGROUND OF THE INVENTION

Regorafenib is a small molecule inhibitor of multiple membrane-bound and intracellular kinases involved in normal cell functions and in pathologic processes such as oncogenesis, tumor angiogenesis, and maintenance of the tumor microenvironment.

Regorafenib is chemically known as 4-[4-({[4-chloro-3-(trifluoromethyl)phenyl]carbamoyl} amino)-3-fluorophenoxy]-N-methylpyridine-2-carboxamide and structurally represented as below.

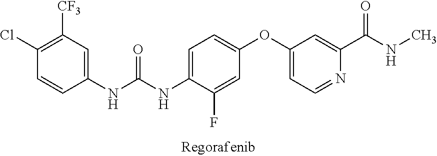 [SMILES: CNC(=O)c1cc(Oc2ccc(NC(=O)Nc3ccc(Cl)c(C(F)(F)F)c3)c(F)c2)ccn1]

Regorafenib is specifically first disclosed in U.S. Pat. No. 8,637,553 and marketed as Regorafenib monohydrate under the brand name STIVAGRA®. It is indicated for the treatment of patients with metastatic colorectal cancer (CRC) who have been previously treated with specific prior therapy.

U.S. Pat. No. 7,351,834 B1 generically discloses Regorafenib, a pharmaceutically acceptable salt thereof, but there is no specific disclosure of Regorafenib in said patent or its equivalents. The patent discloses a process for the preparation of desfluoro analog of Regorafenib i.e. Sorafenib, involving the reaction of 4-chloro-3-(trifluoromethyl)phenylisocyanate with 4-(2-(N-methylcarbamoyl)-4-pyridyloxy)aniline in dichloromethane.

U.S. Pat. No. 8,637,553B2 specifically discloses Regorafenib, pharmaceutically acceptable salts thereof, its composition thereof and the process for the preparation of Regorafenib. In the first step, 4-amino-3-fluorophenol was treated with potassium tert-butoxide and 4-chloro-N-methyl-2-pyridinecarboxamide was added in N,N-dimethylacetamide to form 4-(4-amino-3-fluorophenoxy)pyridine-2-carboxylic acid methylamide which after extraction reacted with 4-chloro-3-(trifluoromethyl)phenylisocyanate in toluene to get regorafenib. The reaction mass was concentrated under reduced pressure and the residue was triturated with diethyl ether. The resulting solid was collected by filtration and dried to afford Regorafenib. The schematic representation is as below:

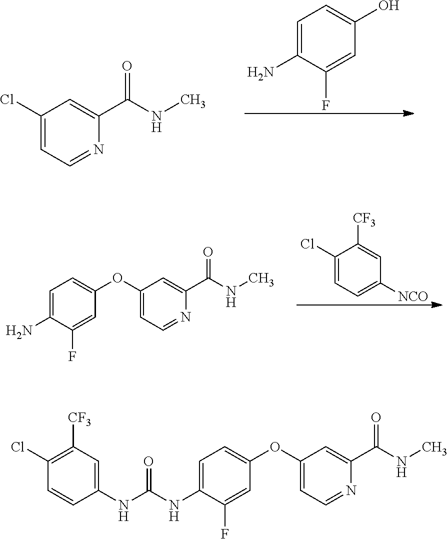

U.S. patent application No. 20060058358 A1 discloses a pharmaceutical composition in the form of a solid dispersion wherein Regorafenib is in substantially amorphous form.

U.S. patent application No. 20100173953 A1 discloses monohydrate of Regorafenib with water content in an amount of 3.6% by weight.

U.S. patent application No. 20100173953 A1 also discloses that the polymorphic form of Regorafenib prepared by the manner described in U.S. Pat. No. 8,637,553B2 corresponds to polymorph I of Regorafenib having a melting point of 186-206° C. and represented its characteristic X-ray diffractogram, IR spectrum, Raman spectrum, FIR spectrum and a ¹³C-solid state-NMR spectrum. As per the disclosure therein monohydrate form has a clearly differentiable X-ray diffractogram, NIR spectrum, FIR spectrum, IR spectrum, ¹³C-solid state NMR spectrum and Raman spectrum to that of polymorph I.

U.S. patent applications, 20100113533 A1 and 20100063112 A1 disclose the polymorph II and polymorph III of Regorafenib, respectively with characteristic X-ray diffraction peaks, melting point and the characteristic IR wave numbers.

PCT publication No. WO2015011659A1 discloses the crystalline polymorphic forms A, B, C+ and D of Regorafenib and processes thereof. This application also discloses the processes for the preparation of polymorph I of Regorafenib. This application mentions the purity of Regorafenib through HPLC but it does not mention about genotoxic impurities of form I.

While the processes disclosed by the prior art are per se effective for Regorafenib, its monohydrate, factors such as purity, product yields, process efficiency, safety and economy are very significant for an industrial scale process of a pharmaceutical product.

The inventors of the present of invention have developed an alternate improved process for the preparation of Regorafenib with high yield and purity. The present process is cost effective and feasible in large scale production also. The present process controls the genotoxic impurities content in final API which can arise from the starting materials.

### SUMMARY OF THE INVENTION

One aspect of the present invention is related to preparation of Regorafenib anhydrous form I, comprising the steps of:
a) reacting 2-fluoronitrobenzene with aluminum powder in presence of aqueous oxalic acid and to get 4-amino-3-fluorophenol,
b) converting 4-amino-3-fluorophenol to 4-(4-amino-3-fluorophenoxy)-N-methylpyridine-2-carboxamide by reacting with 4-Chloro-N-methylpyridine-2-carboxamide,
c) reacting 4-(4-amino-3-fluorophenoxy)-N-methylpyridine-2-carboxamide with 4-chloro-3-(trifluoromethyl)phenylisocyanate in presence of ether solvent to get Regorafenib, d) dissolving Regorafenib obtained from step-c) in ketone solvent and isolation Regorafenib anhydrous form I.

Yet another aspect of the present invention is related to purification of Regorafenib anhydrous form I comprising the steps of:
a) dissolving Regorafenib form I in ketone solvent,
b) isolating Regorafenib anhydrous form I.

### DETAILED DESCRIPTION OF THE INVENTION

The present invention relates to an improved process for the preparation of Regorafenib, wherein reacting 4-(4-amino-3-fluorophenoxy)pyridine-2-carboxylic acid methylamide with 4-chloro-3-(trifluoromethyl)phenylisocyanate in a reaction mixture to get Regorafenib thereafter dissolving Regorafenib in ketone solvent and isolation of Regorafenib anhydrous form I, followed by dissolving in ketone solvent for purification of Regorafenib anhydrous form I.

One embodiment of the present invention is related to preparation of Regorafenib anhydrous form I, comprising the steps of:
a) reacting 2-fluoronitrobenzene with aluminum powder in presence of aqueous oxalic acid and to get 4-amino-3-fluorophenol,
b) converting 4-amino-3-fluorophenol to 4-(4-amino-3-fluorophenoxy)-N-methylpyridine-2-carboxamide by reacting with 4-Chloro-N-methylpyridine-2-carboxamide,
c) reacting 4-(4-amino-3-fluorophenoxy)-N-methylpyridine-2-carboxamide with 4-chloro-3-(trifluoromethyl)phenylisocyanate in presence of ether solvent to get Regorafenib,
d) dissolving Regorafenib obtained from step-c) in ketone solvent and isolation of Regorafenib anhydrous form I.

According to the present invention, 2-Fluoronitrobenzene is added to the solution of Oxalic acid dihydrate in DM water at 25° C. and heated to 80° C. Reducing agent is added to the reaction mass at 80-85° C. and stirred for 90 min. after completion of reaction, reaction mass is cooled to 50° C. Activated carbon is added to the reaction mass, stirred for 30 min and filtered through hyflo bed. The filtrate is washed with ethyl acetate at 40° C., treated with sodium sulfite and adjusted pH to 7.5-8.0 with aqueous ammonia solution. The product is extracted with ethyl acetate, washed with of DM water and concentrated under vacuum at below 50° C. The concentrated mass is stirred in the mixture of ethyl acetate and hexane and filtered the solid. The wet solid was suspended in the mixture of isopropyl alcohol and toluene and added IPA-HCl. The slurry was heated to 50° C. and stirred for 1 h, cooled to 0-5° C. and filtered the solid. The wet solid was dissolved in DM water and adjusted pH to 7.5-8.0 with aqueous ammonia solution at 0-5° C. The solid product was filtered and dried at 40-45° C. to get 4-amino-3-fluorophenol.

Potassium tert-butoxide is added to the solution of 4-amino-3-fluorophenol in N,N-dimethylacetamide at 0° C. and heated to 60° C. 4-Chloro-N-methylpyridine-2-carboxamide is dissolved in N,N-dimethylacetamide and added to the reaction mass at 60° C. The reaction mass is heated to 90° C. and stirred for 90 min. After completion reaction, reaction mass is cooled to 30° C., added into DM water, stirred for 60 min, filtered the solid product and dried. The dried product is dissolved in ethyl acetate at 70° C., treated with activated carbon for 30 min and filtered through hyflo bed. The filtrate was partially concentrated, cooled to 0-5° C. and filtered the solid and dried to get 4-(4-amino-3-fluorophenoxy)-N-methylpyridine-2-carboxamide.

4-Chloro-3-(trifluoromethyl)phenylisocyanate is dissolved in ether solvent and added to the solution of 4-(4-amino-3-fluorophenoxy)-N-methylpyridine-2-carboxamide in ether solvent at 30° C. and stirred for 12 h. The reaction is monitored by LC-MS analysis and controlled 4-(4-amino-3-fluorophenoxy)-N-methylpyridine-2-carboxamide content to NMT 50 ppm during reaction. The reaction mass is concentrated and co-distilled with acetone. The concentrated mass was dissolved in ketone solvent at 55° C., treated with activated carbon for 30 min and filtered through hyflo bed. The filtrate was partially concentrated, cooled to 20° C., filtered and dried the product to yield crude Regorafenib anhydrous form I.

According to the present invention, reducing agent is selected from zinc or aluminum, preferably aluminum powder.

According to the present invention, ether solvent is selected from diethyl ether, 2-methyl tetrahydrofuran, tetrahydrofuran, preferably tetrahydrofuran.

According to the present invention, ketone solvent is selected from acetone, methylethylketone and methylisobutylketone, preferably acetone.

Yet another embodiment of the present invention is related to purification of Regorafenib anhydrous form I comprising the steps of:
a) dissolving Regorafenib form I in ketone solvent,
b) isolating Regorafenib anhydrous form I.

According to the present invention, crude Regorafenib form I is dissolved in ketone solvent at 55° C., treated with activated carbon for 30 min and filtered through hyflo bed. The filtrate was partially concentrated, cooled to 20° C. and the solid product was filtered and dried at 50-55° C. to yield pure Regorafenib anhydrous form I.

According to the present invention, ketone solvent is selected from acetone, methylethylketone and methylisobutylketone, preferably acetone.

According to the present invention, 4-(4-amino-3-fluorophenoxy)-N-methylpyridine-2-carboxamide is a potentially genotoxic impurity. This genotoxic impurity content is monitored by LC-MS during reaction and controlled to not more than 50 ppm in the reaction mass and also in crude Regorafenib anhydrous form I, and 20 ppm in final Regorafenib anhydrous form I. This potentially genotoxic impurity limit is achieved in Regorafenib through the controlled reaction of 4-(4-amino-3-fluorophenoxy)-N-methylpyridine-2-carboxamide with 4-chloro-N-methylpyridine-2-carboxamide in tetrahydrofuran for longer hours followed by purification in acetone solvent.

### Advantages of the Present Invention

This genotoxic impurity is very well controlled in final API.
The present process is cost effective and commercially feasible process in large scale.

The following examples are provided for illustrative purpose only and are not intended to limit the scope of invention in anyway.

### EXPERIMENTAL SECTION

### Example-1: Preparation of 4-amino-3-fluorophenol

2-Fluoronitrobenzene (300 g) was added to the solution of oxalic acid dihydrate (858 g) in DM water (7.5 L) at 25° C. and heated to 80° C. Aluminum powder (98.8 g) was added to the reaction mass at 80-85° C. and stirred for 90 min. After completion of reaction, reaction mass was cooled to 50° C. Activated carbon (30 g) was added to the reaction mass, stirred for 30 min and filtered through hyflo bed. The filtrate was washed with ethyl acetate (2×1500 ml) at 40° C., treated with sodium sulfite (300 g) and adjusted pH to 7.5-8.0 with aqueous ammonia solution. The product was extracted with ethyl acetate (2×1500 ml), washed with DM water (300 ml) and concentrated under vacuum at below 50° C. The concentrated mass was stirred in the mixture of ethyl acetate (60 ml) and hexane (1140 ml) and filtered the solid. The wet solid was suspended in the mixture of isopropyl alcohol (150 ml) and toluene (600 ml) and added IPA-HCl (198 g, 24% w/w). The slurry was heated to 50° C. and stirred for 1 h, cooled to 0-5° C. and filtered the solid. The wet solid was dissolved in DM water (700 ml) and adjusted pH to 7.5-8.0 with aqueous ammonia solution at 0-5° C. The solid product was filtered and dried at 40-45° C. (109.9 g; 40.7%). HPLC purity: 99.867%

### Example-2: Preparation of 4-(4-Amino-3-fluorophenoxy)-N-methylpyridine-2-carboxamide

Potassium tert-butoxide (90 g) was added to the solution of 4-amino-3-fluorophenol (90 g) in N,N-dimethylacetamide (400 ml) at 0° C. and heated to 60° C. 4-Chloro-N-methylpyridine-2-carboxamide (100 g) was dissolved in N,N-dimethylacetamide (100 ml) and added to the reaction mass at 60° C. The reaction mass was heated to 90° C. and stirred for 90 min. After completion of reaction, reaction mass was cooled to 30° C. The reaction mass was slowly added into DM water (2500 ml), stirred for 60 min, filtered the solid product and dried. The dry product was dissolved in ethyl acetate (1200 ml) at 70° C., treated with activated carbon (12 g) for 30 min and filtered through hyflo bed. The filtrate was partially concentrated, cooled to 0-5° C. and filtered the solid and dried (92.9 g, theory yield: 50.2%).

HPLC purity: 98.893%

### Example-3: Preparation of 4-[4-({[4-chloro-3-(trifluoromethyl) phenyl] carbamoyl} amino)-3-fluorophenoxy]-N-methylpyridine-2-carboxamide (Crude Regorafenib)

4-Chloro-3-(trifluoromethyl)phenylisocyanate (101.8 g) was dissolved in THF (200 ml) and added to the solution of 4-(4-amino-3-fluorophenoxy)-N-methylpyridine-2-carboxamide in THF (800 ml) at 30° C. and stirred for 12 h. The reaction was monitored by LC-MS analysis and controlled 4-(4-amino-3-fluorophenoxy)-N-methylpyridine-2-carboxamide content to NMT 50 ppm during reaction. The reaction mass was concentrated and co-distilled with acetone (2×200 ml). The concentrated mass was dissolved in acetone (4000 ml) at 55° C., treated with activated carbon (15 g) for 30 min and filtered through hyflo bed. The filtrate was partially concentrated, cooled to 20° C., filtered and dried the product to yield crude Regorafenib anhydrous form I (135.9 g, 73.46%). HPLC purity: 99.431%

### Example-4: Preparation of Pure Regorafenib Anhydrous Form I

Crude Regorafenib anhydrous form I (130 g) was dissolved in acetone (2600 ml) at 55° C., treated with activated carbon (15 g) for 30 min and filtered through hyflo bed. The filtrate was partially concentrated, cooled to 20° C. and the solid product was filtered and dried at 50-55° C. to yield pure Regorafenib anhydrous form I (106.5 g; 81.9%).

HPLC purity: 99.737%.

We claim:

1. A process for the preparation of Regorafenib anhydrous form I, 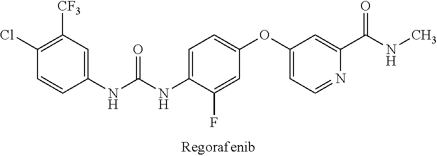 [SMILES: CNC(=O)c1cc(Oc2ccc(NC(=O)Nc3ccc(Cl)c(C(F)(F)F)c3)c(F)c2)ccn1]
comprising the steps of:
a) reacting 2-fluoronitrobenzene with oxalic acid in presence of a reducing agent at elevated temperature to get 4-amino-3-fluorophenol,
b) converting 4-amino-3-fluorophenol to 4-(4-amino-3-fluorophenoxy)-N-methylpyridine-2-carboxamide by reacting with 4-chloro-N-methylpyridine-2-carboxamide in presence of a base in a polar solvent at elevated temperature,
c) reacting 4-(4-amino-3-fluorophenoxy)-N-methylpyridine-2-carboxamide with 4-chloro-3-(trifluoromethyl)phenylisocyanate in presence of ether solvent to get Regorafenib,
d) dissolving regorafenib obtained from step c) in ketone solvent and isolation of regorafenib anhydrous form I.

2. The process according to claim 1, wherein reducing agent is selected from zinc and aluminum powder.

3. The process according to claim 1, wherein the elevated temperature used in step a) is 40-90° C.

4. The process according to claim 1, wherein the base used in step b) is selected from sodium or potassium hydroxide, sodium or potassium t-butoxide potassium carbonate.

5. The process according to claim 1, wherein the polar solvent used in step b) is selected from DMF, DMAc, THF.

6. The process according to claim 1, wherein the temperature of the reaction is 40-100° C.

7. The process according to claim 1, wherein ether solvent used in step c) is selected from diethyl ether, 2-methyl tetrahydrofuran, tetrahydrofuran.

8. The process according to claim 1, wherein the ketone solvent used in step d) is selected from acetone, methyl ethyl ketone and methyl isobutyl ketone.

9. A process for the purification of Regorafenib anhydrous form I comprising the steps of:
a) dissolving Regorafenib form I in a ketone solvent,
b) isolating Regorafenib anhydrous form I.

10. The process according to claim 9, wherein ketone solvent is selected from acetone, methyl ethyl ketone and methyl isobutyl ketone, preferably acetone.

11. Regorafenib anhydrous form I having 4-(4-amino-3-fluorophenoxy)-N-methylpyridine-2-carboxamide, which is a potentially genotoxic impurity content less than 50 ppm.

12. The Regorafenib according to claim 11, having 4-(4-amino-3-fluorophenoxy)-N-methylpyridine-2-carboxamide, which is a potentially impurity content less than 20 ppm.
